(19) United States
(12) Patent Application Publication
(10) Pub. No.: US 2013/0310904 A1
(43) Pub. Date: Nov. 21, 2013

(54) PHOTOTHERAPEUTIC APPARATUS

(71) Applicant: Song Kyo Seo, Daegu-si (KR)

(72) Inventor: Song Kyo Seo, Daegu-si (KR)

(21) Appl. No.: 13/983,780

(22) PCT Filed: Feb. 2, 2012
(86) PCT No.: PCT/KR2012/000802

§ 371 Date: Aug. 6, 2013

(30) Foreign Application Priority Data
Feb. 8, 2011 (KR) 10-2011-0011067

(51) Int. Cl. A61N 5/06

(52) U.S. Cl. CPC A61N 5/06
USPC 607/89; 607/88

(57) ABSTRACT

Disclosed is a phototherapeutic apparatus, which includes a net consisting of a strand of optical fiber; a connector connected to one end of the optical fiber; and a radiation device connected to the connector. The net may be easily attached onto a wig, hat, underwear, or the like, thus enabling a user to unconsciously receive a phototherapy during normal daily life.

### TECHNICAL FIELD

The present invention relates to a phototherapeutic apparatus, and more particularly, to a phototherapeutic apparatus invented so that light helpful for human body is irradiated to skin through a net of optical fiber.

### BACKGROUND ART

Recently, there are many cases in which radiating light to a skin of a human body is used as a treatment for a disease and improvement of a physical constitution.

As the light used for a therapy, a low output laser or an LED light has been in the spotlight.

The low output laser is a laser with a frequency of 1 to 500 nm, and it had been studied that a He—Ne laser of a frequency of 638.8 nm by the middle of 1980's, and a diode laser of a red region (630 to 670 nm) and an infrared ray region (830 to 1300 nm) by the end of 1990's have an effect of facilitating bio-activation, and the low output laser is turned out to have a bio-activation effect, such as a supply of nutrition to tissues and a regeneration of tissues, according to an anti-inflammatory effect, activation of an immune system, protection of blood vessels, and an enhancement of a function of microcirculation of blood.

Further, the low output laser has been medically proven to expand blood vessels and improve blood circulation to help restore damaged cell tissues to its original states when the low output laser is radiated to a human body, and to have an effect of stimulation, such as acupuncture or moxa cautery, to alleviate skin or muscle pain or facilitate complete recovery of an affected area when the low output laser is radiated to acupoints.

The LED light generates hormone (serotonin) that serves to generate collagen and elastin, discharge active humoral factor exhibiting an anti-tumor effect, expand blood vessels, and contract blood vessel by stimulating macrophage and fibroblasts.

Further, the LED light generates glycosaminoglycan (GAG) by stimulating msst cells and generates proteoglycan (a compound in which protein is covalently bonded to a glycosaminoglycan chain) by bonding the glycosaminoglycan chain to protein. The proteoglycan forms a cell material of a higher animal by forming a macromolecular aggregate together with hyaluronic acid and collagen.

As described above, both the low output laser and the LED light are light helpful for the human body, and have been certified from the Food and Drug Administration (FDA), and are widely used for a skin pore therapy, acne therapy, a therapy of a damage by sunlight, a generation of collagen, a glow therapy, and wrinkle therapy.

In order to radiate the low output laser and the LED light to skin, a product, in which a user attaches a light emitting diode radiating the light to a tip end of a bar to radiate the light to the whole body by holding the bar, has been developed and already released on the market.

However, since such the aforementioned medial appliance can radiate light to the skin only during a phototherapy, people busy with their daily lives are restricted by time, and limited to receiving therapy over a long period of time. In general, it is known that a therapy effect is exhibited when the light is radiated to the skin 3 hours or more per day for 30 days. However, there is a problem in that people who are busy find it difficult to receive therapy on a daily basis.

For example, the low output laser and the LED light have been already clinically verified to have special efficacy in stimulating hair growth of a bald head, but a lot of time is taken for daily performing a phototherapy on a scalp, so that many people receiving a phototherapy have frequently abandoned the therapy midway.

Accordingly, an object of the present invention is to provide a phototherapeutic apparatus configured to enable people to receive a phototherapy to unconsciously receive radiation of a laser or LED light on the skin during normal daily life over a long period of time.

### DISCLOSURE

### Technical Problem

In order to radiate the low output laser and the LED light, a product, in which a light emitting diode radiating the light is attached to an end of a bar to allow a user to radiate the light to the whole body by holding the bar, has been developed and already released on the market.

However, even though such a medical appliance can radiate light to the skin, people busy in their daily lives are restricted by time, and limited to receiving therapy over a long period of time. In general, it is known that a treatment effect is exhibited when the light is radiated to the skin 3 hours or more per day for 30 days. However, there is a problem in that people find it difficult to have therapy on a daily basis due to the amount of time required.

For example, the low output laser and the LED light have been already clinically verified to have special efficacy in stimulating hair growth of a bald head, but a lot of time is taken for daily performing a phototherapy on a scalp, so that many people to receive the phototherapy have frequently abandoned the therapy midway.

Accordingly, an object of the present invention is to provide a phototherapeutic apparatus configured to enable people to receive phototherapy by unconsciously receiving radiation of a laser or LED light on the skin during normal daily life over a long period of time.

### Technical Solution

A phototherapeutic apparatus according to the present invention includes: a net consisting of a strand of optical fiber; a connector connected to one end of the optical fiber; and a radiation device connected to the connector.

Further, the radiation device includes a light emitting diode in which a lamp is fitted to the connector to radiate light, a switch connected to the light emitting diode to manage on/off of electricity, and a battery connected to supply power to the switch.

Further, the light emitting diode is a laser diode generating a low output laser.

Further, the light emitting diode is an LED diode.

Further, a sheet is attached onto one surface of the net.

Further, the optical fiber is provided with a coating layer in a longitudinal direction, and the coating layer is opened toward one side in the longitudinal direction.

Further, the radiation device further includes a controller configured to turn on/off the switch by receiving an on/off signal, a receiver configured to receive the on/off signal through radio waves, and transmit the on/off signal to the controller, and a transmitter configured to transmit the on/off signal to the receiver through radio waves.

### Advantageous Effects

According to the phototherapeutic apparatus according to the present invention, the net may be easily attached to a wig, a hat, or underwear, thereby achieving an effect in that a user unconsciously receives a phototherapy during normal daily life. That is, the inconvenience of intentionally sparing time for the therapy is removed.

Accordingly, a person to receive a phototherapy continuously receives the phototherapy, thereby achieving a desired therapeutic effect.

### DESCRIPTION OF DRAWINGS

FIG. 1 is a front view illustrating a phototherapeutic apparatus according to a technology of the present invention.

FIG. 2 is a side view of FIG. 1 viewed in direction V, and is a side view illustrating a state in which a sheet is attached to one surface of a net of optical fiber.

FIG. 3 is a cross-sectional view illustrating a state in which a light emitting diode of a radiation device is connected to the optical fiber in the phototherapeutic apparatus according to the technology of the present invention.

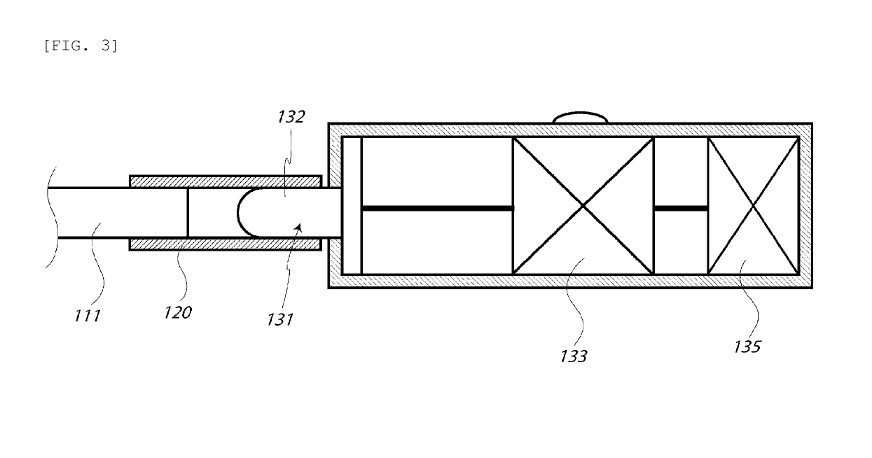

FIG. 4 is a perspective view illustrating an example in which the phototherapeutic apparatus according to the technology of the present invention is applied to a scalp.

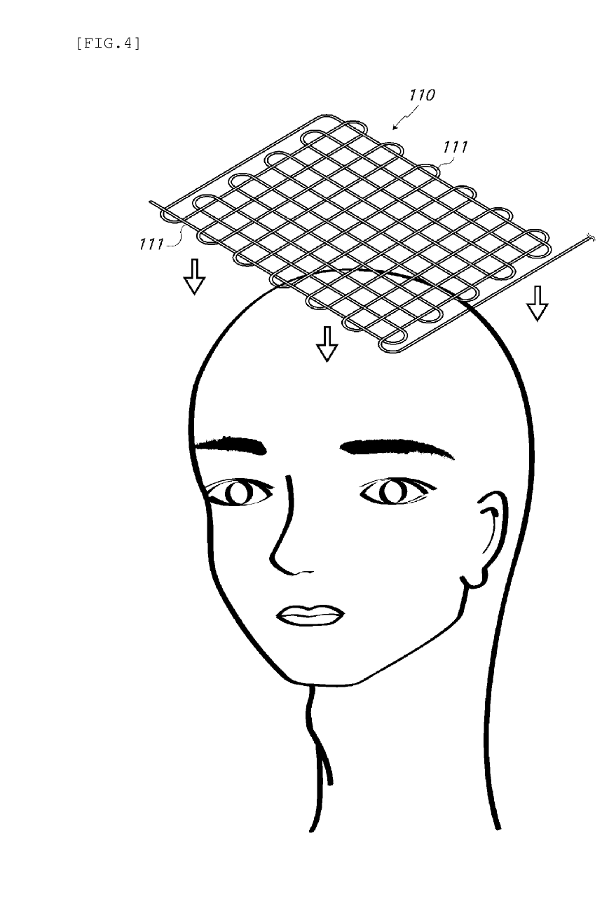

FIG. 5 is a block diagram illustrating a circuit of the radiation device of the phototherapeutic apparatus according to the technology of the present invention.

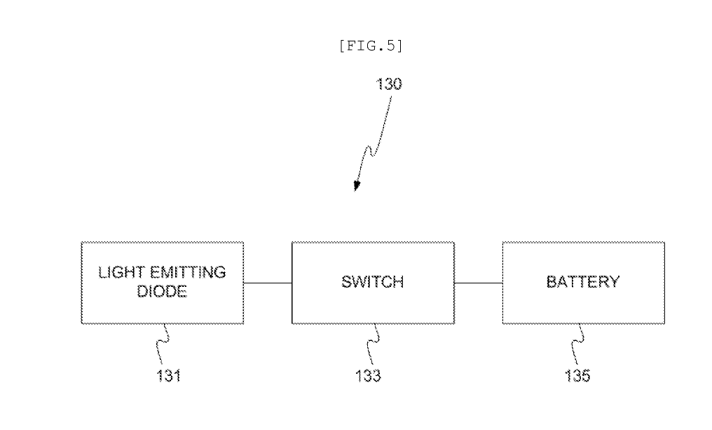

FIG. 6 is a front view illustrating another embodiment of a net of optical fiber used in the radiation device of the phototherapeutic apparatus according to the technology of the present invention, in which a thread is knotted in the optical fiber.

FIG. 7 is a cross-sectional view illustrating another embodiment of the optical fiber configured in the phototherapeutic apparatus according to the technology of the present invention.

FIG. 8 is a block diagram illustrating an example of a circuit configured so that the radiation device of the phototherapeutic apparatus according to the technology of the present invention is remotely controlled.

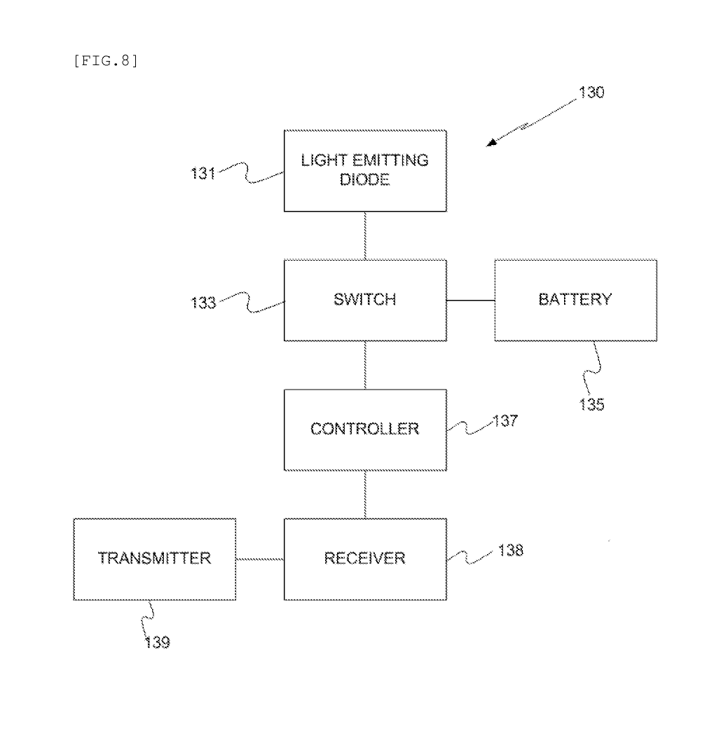

FIG. 9 is a front view illustrating another embodiment of a net of optical fiber used in the radiation device of the phototherapeutic apparatus according to the technology of the present invention, in which a plurality of strands of optical fiber is integrally connected.

### BEST MODE

In general, a phototherapy of radiating light, such as a low output laser and an LED light, helpful for a human body has been certified from the Food and Drug Administration (FDA), and is widely used for a skin pore therapy, an acne therapy, a therapy of a damage by sunlight, a generation of collagen, a glow treatment, and a wrinkle treatment.

The present invention is characterized in that a person to receive a phototherapy is capable of unconsciously and easily receiving the light, such as the low output laser and the LED light, helpful for a human body.

### MODE FOR CARRYING OUT THE INVENTION

A phototherapeutic apparatus includes a net 110 consisting of a strand of optical fiber 111, a connector 120 connected to one end of the optical fiber 111, and a radiation device 130 connected to the connector 120 to radiate light.

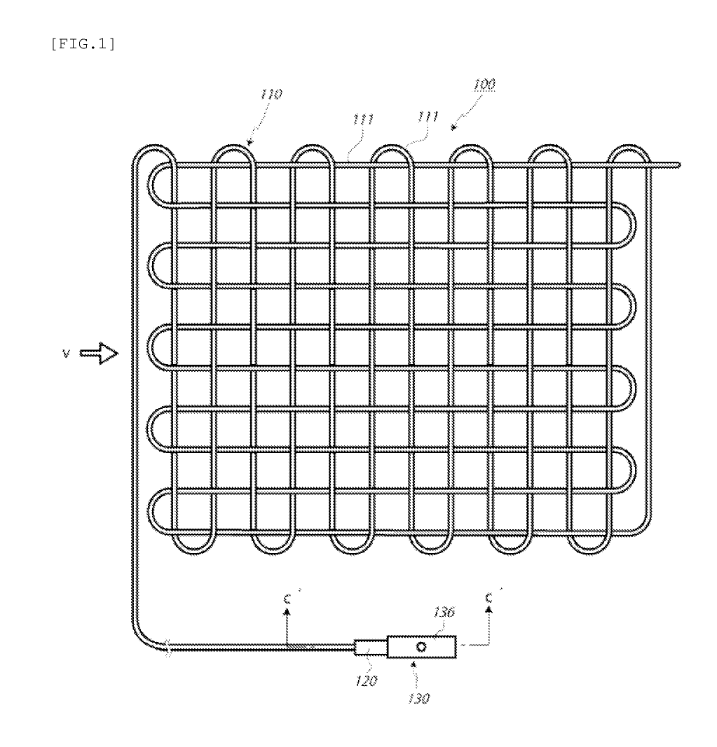

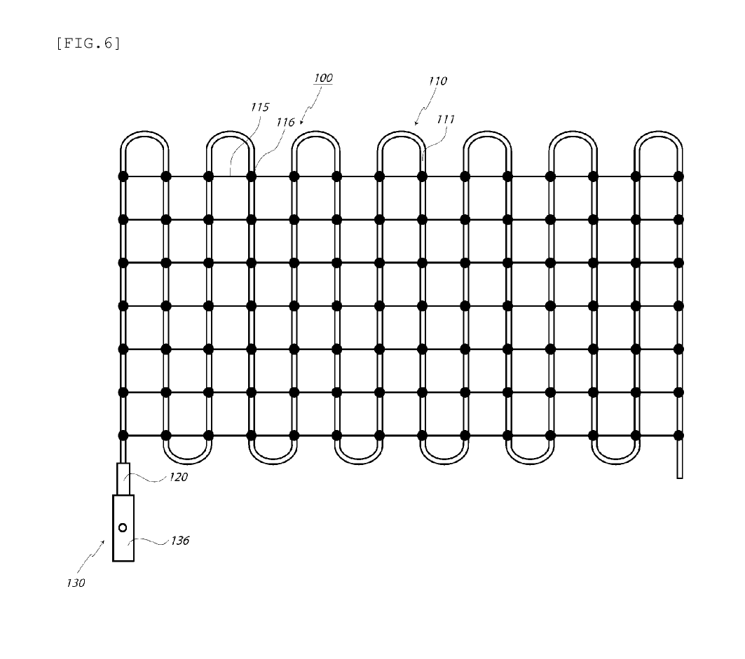

The net 110 is formed by weaving the strand of optical fiber 111 into a fabric, and as a example, as illustrated in FIG. 1, the strand of optical fiber 111 is bent in zigzags several times to form a warp a, and then a weft b connected to the warp a passes through the warp b in zigzags to form the net. Otherwise, as another example, as illustrated in FIG. 6, the strand of optical fiber 111 is bent in zigzags several times to form a warp a, and then a thread 115 is inserted into the warp a in a direction of a weft, so that a knot 116 is tied in each warp a.

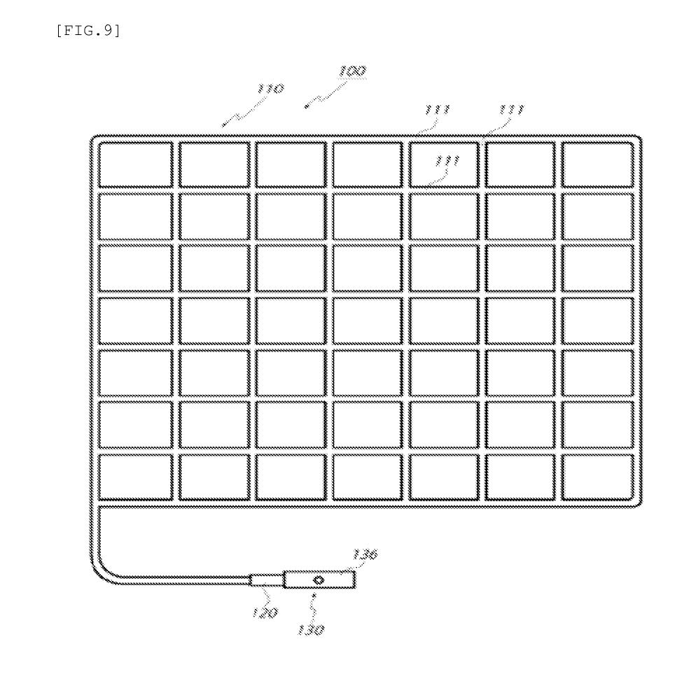

As yet another example of the net 110, as illustrated in FIG. 9, the plurality of strands of optical fiber 111 may be integrally connected with each other to be formed as the net 110, and for example, the strands of optical fiber 111 may be vertically or horizontally arrange, and each strand of optical fiber 111 may be connected to another strand of optical fiber 111, so that the strands of optical fiber 111 may be integrally formed. In this case, the strand of optical fiber 111 is drawn long so as to be connected with the connector 120, and the radiation device 130 is connected to the connector 120.

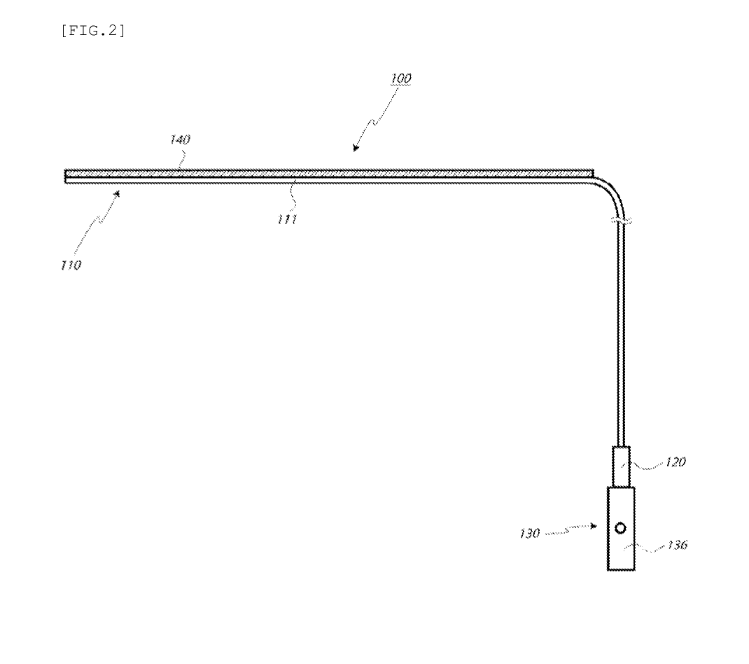

Further, as illustrated in FIG. 2, the net 110 may be configured so that light is radiated toward the skin only in one direction by attaching a sheet 140 onto one surface thereof. As an example, textile, plastic, or paper, which may prevent light from being transmitted, is available as the sheet 140.

The optical fiber 111 uniformly emits light from a side surface along the optical fiber, and is generally and widely used for optical fiber illumination.

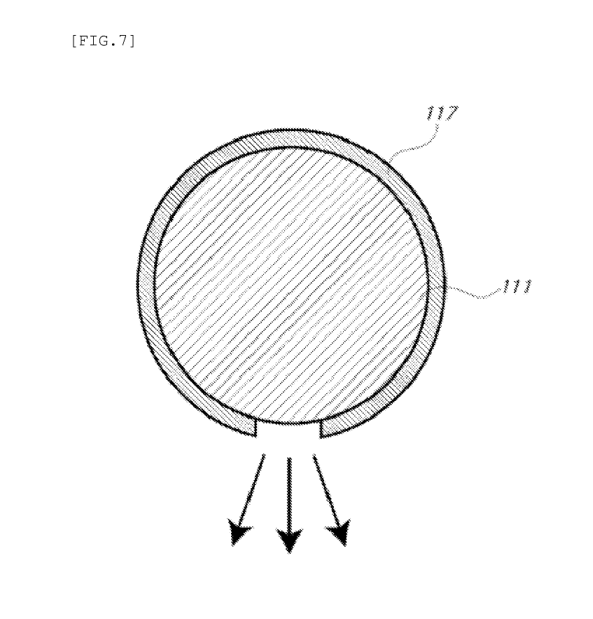

In the present invention, in order to make the light of the optical fiber 111 be radiated toward the skin only in one direction, as illustrated in FIG. 7, a coating layer 117 is formed in a longitudinal direction, and the coating layer 117 is opened toward one side in the longitudinal direction, so that the light may be radiated only in one direction. It is a matter of course that the light is uniformly radiated in the longitudinal direction.

The connector 120 has, for example, a shape of a pipe fitted to one end 113 of the optical fiber 111, and may be formed in a dark color so as to prevent light from leaking.

The radiation device 130 includes a light emitting diode 131 in which a lamp 132 is fitted to the connector 120 to radiate light helpful for a human body, a switch 133 connected to the light emitting diode 131 to manage on/off of electricity, and a battery 135 connected to supply power to the switch 133. For example, a laser diode radiating a low output laser or an LED diode is available as the light emitting diode 131. Further, the radiation device 130 is accommodated in a casing 136 so as not to be damaged by external force.

The radiation device 130 includes a controller 137 for turning on/off the switch 133 by receiving an on/off signal so that the radiation device 130 may be remotely controlled, a receiver 138 for receiving the on/off signal through radio waves, and transmitting the on/off signal to the controller 137, and a transmitter 139 for transmitting the on/off signal to the receiver 138 through radio waves. The transmitter 139 may be served with a typical remote controller.

A usage example of the present invention according to the configuration will be described below.

First, a bald person to receive a phototherapy attaches the net 110 to a wig or hat, and then puts the wig or the hat on a head. Then, the person operates the switch 133 so as for light to be radiated from the light emitting diode 131. Then, the light is transmitted along the strand of optical fiber 111 consisting of the net 110, and the light is radiated toward a scalp. In this case, the light radiated to the skin may be concentrated by the coating layer 117. Further, the aforementioned advantage may be achieved even by the sheet 140 as a matter of course. Further, as illustrated in FIG. 9, even in a case where the plurality of strands of optical fiber 111 is integrally connected, when light is radiated to the long drawn strand of optical fiber 111, the light is also transmitted through each optical fiber 111 as a matter of course.

In addition, when the net 110 is attached onto an internal surface of underwear, there is an effect of naturally receiving a phototherapy during normal daily life.

Further, in a case where the transmitter 139 and the receiver 139 are provided, the present invention may be remotely controlled as a matter of course.

### INDUSTRIAL APPLICABILITY

According to the present invention, the net 110 may be easily attached to a wig, a hat, or underwear, thereby achieving an advantage in that a user unconsciously receives a phototherapy during normal daily life.

Accordingly, a person to receive a phototherapy continuously receives the phototherapy, thereby achieving a desired therapeutic effect.

1-8. (canceled)

9. A phototherapeutic apparatus, comprising:
a net consisting of a strand of optical fiber;
a connector connected to one end of the optical fiber; and
a radiation device connected to the connector.

10. A phototherapeutic apparatus, comprising:
a net formed by integrally connecting a plurality of strands of optical fiber;
a connector connected to the drawn optical fiber among the plurality of strands of optical fiber ; and
a radiation device connected to the connector.

11. The phototherapeutic apparatus of claim 9, wherein the radiation device includes a light emitting diode in which a lamp is fitted to the connector to radiate light, a switch connected to the light emitting diode to manage on/off of electricity, and a battery connected to supply power to the switch.

12. The phototherapeutic apparatus of claim 11, wherein the light emitting diode is a laser diode generating a low output laser.

13. The phototherapeutic apparatus of claim 11, wherein the light emitting diode is an LED diode.

14. The phototherapeutic apparatus of claim 10, wherein the radiation device includes a light emitting diode in which a lamp is fitted to the connector to radiate light, a switch connected to the light emitting diode to manage on/off of electricity, and a battery connected to supply power to the switch.

15. The phototherapeutic apparatus of claim 14, wherein the light emitting diode is a laser diode generating a low output laser.

16. The phototherapeutic apparatus of claim 14, wherein the light emitting diode is an LED diode.

17. The phototherapeutic apparatus of claim 9, wherein a sheet is attached onto one surface of the net.

18. The phototherapeutic apparatus of claim 10, wherein a sheet is attached onto one surface of the net.

19. The phototherapeutic apparatus of claim 9, wherein the optical fiber is provided with a coating layer in a longitudinal direction, and the coating layer is opened toward one side in the longitudinal direction.

20. The phototherapeutic apparatus of claim 10, wherein the optical fiber is provided with a coating layer in a longitudinal direction, and the coating layer is opened toward one side in the longitudinal direction.

21. The phototherapeutic apparatus of claim 9, wherein the radiation device further includes a controller configured to turn on/off the switch by receiving an on/off signal, a receiver configured to receive the on/off signal through radio waves, and transmit the on/off signal to the controller, and a transmitter configured to transmit the on/off signal to the receiver through radio waves.

22. The phototherapeutic apparatus of claim 10, wherein the radiation device further includes a controller configured to turn on/off the switch by receiving an on/off signal, a receiver configured to receive the on/off signal through radio waves, and transmit the on/off signal to the controller, and a transmitter configured to transmit the on/off signal to the receiver through radio waves.
